(19) United States
(12) Patent Application Publication
(10) Pub. No.: US 2025/0292926 A1
(43) Pub. Date: Sep. 18, 2025

(54) METHOD FOR TREATING RADIOACTIVE WASTE USING APTAMERS

(71) Applicant: INDUSTRY ACADEMY COOPERATION FOUNDATION OF SEJONG UNIVERSITY, Seoul (KR)

(72) Inventors: MI YONG YUN, Seoul (KR); SUN YOUNG LEE, Gangwon-do (KR); KYOON HO CHA, Seoul (KR)

(21) Appl. No.: 18/981,923

(22) Filed: Dec. 16, 2024

(30) Foreign Application Priority Data
Dec. 15, 2023 (KR) 10-2023-0183816

(51) Int. Cl. G21F 9/12

(52) U.S. Cl. CPC G21F 9/12

(57) ABSTRACT

A method for treating radioactive liquid waste using aptamer includes passing radioactive liquid waste including radioactive metal ions through a column filled with aptamer-bound beads to combine the metal ions with the aptamer, performing denaturation of the aptamer to separate the radioactive metal ions from the column, and reusing the column to treat radioactive liquid waste, such that specific metal elements in the radioactive liquid waste may be efficiently isolated while easily separating the radioactive metal ions bound to the column, thereby enabling the column to be reused.

### CROSS-REFERENCE TO RELATED APPLICATION AND CLAIM OF PRIORITY

This application claims the benefit under 35 USC § 119 of Korean Patent Application No. 10-2023-0183816, filed on Dec. 15, 2023, in the Korean Intellectual Property Office, the entire disclosure of which is incorporated herein by reference for all purposes.

### REFERENCE TO AN ELECTRONIC SEQUENCE LISTING

A sequence listing electronically submitted on Dec. 16, 2024 as a XML file named 20241216_LC1022431_TU_SEQ.XML, created on Dec. 16, 2024 and having a size of 18,449 bytes, is incorporated herein by reference in its entirety.

### BACKGROUND

### 1. Field of the Invention

The present invention relates to a method for treating radioactive liquid waste using aptamer.

### 2. Description of the Related Art

Radioactive ionic materials generated during operation exist in cooling water in a primary cooling system and a spent fuel pool storage of a nuclear facility, and an ion exchange resin method is usually used to remove the above materials. For this reason, among waste from the operation of the existing nuclear facilities, a great amount of ion exchange resin used for removing the radioactive ionic materials as well as the waste generated during production of nuclear power itself are classified into radioactive waste and result in mass production of secondary waste.

Radioactive waste management standards and waste amounts are different by country, therefore, there is a need for development of radioactive waste processing techniques matching with domestic requirements rather than direct introduction of foreign techniques. In particular, Korea has a high population as a percentage of the nation's territory such that it is difficult to increase radioactive waste disposal facilities, in addition, since a high level of safety management for radioactive waste is required, economic and social costs for radioactive waste disposal are very expensive.

Particularly, since cesium, strontium, antimony, etc. among the radioactive materials generated during the operation are not removed well from the primary cooling water of a nuclear reactor and laundry waste water of the nuclear reactor by the existing ion exchange resin method, a separate adsorbent for removing and other high-priced equipment have been used. Accordingly, not only the existing ion exchange resin but also radioactive waste is further mass-produced whereby it pays high expenses for removing the same.

The present patent invention is aimed to construct a novel process system capable of replacing the existing radioactive ion treatment process. In particular, a radioactive ion removing technique using aptamer with a low facility unit cost and expense would be developed thus to save waste disposal costs in addition to further reducing the weight of waste.

### SUMMARY

An object of the present invention is to prove an aptamer-bead column for treating radioactive liquid waste.

Another object of the present invention is to provide a system for treating radioactive liquid waste including an aptamer-bead column and a treatment method thereof.

To achieve the above objects, the following technical solutions are adopted in the present invention.

1. A method for treating radioactive liquid waste using aptamer, including: passing radioactive liquid waste including radioactive metal ions through a column filled with aptamer-bound beads to combine the metal ions with the aptamer; performing denaturation of the aptamer to separate the radioactive metal ions from the column; and reusing the column to treat radioactive liquid waste.

2. The method for treating radioactive liquid waste using aptamer according to the above 1, wherein the denaturation is performed by passing hot water at 50 to 100° C. through the column.

3. The method for treating radioactive liquid waste using aptamer according to the above 2, further including denaturation of the aptamer by reusing the hot water.

4. The method for treating radioactive liquid waste using aptamer according to the above 1, further including renaturation of the denaturated aptamer at room temperature by adding a buffer to the column.

5. The method for treating radioactive liquid waste using aptamer according to the above 1, wherein the aptamer having a length of 30 to 150 nt.

6. The method for treating radioactive liquid waste using aptamer according to the above 1, wherein the aptamer has any one nucleotide sequence of SEQ ID NOs: 1 to 18.

7. The method for treating radioactive liquid waste using aptamer according to the above 1, wherein the bead has a diameter of 0.5 μm to 1 cm.

8. The method for treating radioactive liquid waste using aptamer according to the above 1, wherein the aptamer is bound to the bead in an amount of 0.1 nmol to 10 mmol per 1 ml unit volume of the bead.

9. The method for treating radioactive liquid waste using aptamer according to the above 1, wherein the aptamer is bound to the bead by a bond of biotin and any one selected from the group consisting of streptavidin, an amine group, a carboxyl group, a hydroxyl group, a sulfone group, a dimethylamine group and an epoxy group.

10. An aptamer-bead column for treating radioactive liquid waste, including beads having a diameter of 0.5 μm to 1 cm filled therein, to which aptamer having a length of 30 to 150 nt is bound.

11. The aptamer-bead column for treating radioactive liquid waste according to the above 10, wherein the aptamer is bound to the bead by a bond of biotin and any one selected from the group consisting of streptavidin, an amine group, a carboxyl group, a hydroxyl group, a sulfone group, a dimethylamine group and an epoxy group.

12. The aptamer-bead column for treating radioactive liquid waste according to the above 10, wherein the aptamer is bound to the bead in an amount of 0.1 nmol to 10 mmol per 1 ml unit volume of the bead.

13. The aptamer-bead column for treating radioactive liquid waste according to the above 10, wherein the aptamer has any one nucleotide sequence of SEQ ID NOs: 1 to 18.

The aptamer-bead column for treating radioactive liquid waste according to the present invention has high selectivity to specific ionic elements.

The aptamer-bead column for treating radioactive liquid waste according to the present invention may be effectively utilized when specific metal elements in the radioactive liquid waste have high concentrations.

The aptamer-bead column for treating radioactive liquid waste according to the present invention may easily separate radioactive metal ions thereby enabling the column to be reused.

The aptamer-bead column for treating radioactive liquid waste according to the present invention is effective in volume reduction of radioactive liquid waste and saving disposal costs of the radioactive waste.

The method for treating radioactive liquid waste including the aptamer-bead column of the present invention has high selectivity to specific ionic elements such that the above method may be effectively utilized when specific metal elements in the radioactive liquid waste have high concentrations, and radioactive metal ions bound to the column may be easily separated thereby enabling the column to be reused, such that the inventive method is effective in volume reduction of radioactive liquid waste and saving disposal costs of the radioactive waste.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view illustrating a process for treatment of radioactive liquid waste using aptamers according the present invention;

FIG. 2 illustrates a method of determining an optimal combining ratio of aptamer and bead;

FIGS. 3A to 3D are experimental data confirming the optimal combining ratio of aptamer and bead using a flow cytometer and a fluorescent microscope;

FIG. 4 illustrates an ion removal rate according to usage of aptamer when an amount of aptamer to usage of bead is used in minimum 1/512 times to maximum 4 times based on the optimal rate, wherein it was confirmed that the ion removal rate is the highest when the optimal rate is used, and the number of beads and aptamers practically used in the above experiment is described in Table 3;

FIGS. 5A and 5B illustrate results of assessment for possible reuse of the aptamer-bead column as well as the performance thereof, wherein FIG. 5A is a target ion removal rate of the aptamer, and FIG. 5B is a target ion collection rate;

FIG. 6 A illustrates reuse protocol of the column, and FIG. 6B illustrates the removal rate according to the number of reuses;

FIG. 7 illustrates results of confirming that the ion bound to the aptamer is concentrated, and showing that hot water can be reused;

FIG. 8 is the result of evaluation showing target ion selectivity of the representative aptamer-bead column;

FIG. 9 is the result of executing field tests with regard to the sample including samples containing the radioactive material in the Korea Atomic Energy Research Institute (KAERI), and primary cooling water of an actual nuclear power plant; and

FIG. 10 illustrates a reuse manner using hot water in briefly the method of recycling aptamers according to the present invention.

### DETAILED DESCRIPTION OF THE INVENTION

The present invention provides an aptamer-bead column for treating radioactive liquid waste, and a method for treating radioactive liquid waste using the column.

The present invention provides a method for treating radioactive liquid waste using aptamer, including: passing radioactive liquid waste including radioactive metal ions through a column filled with aptamer-bound beads to combine the metal ions with the aptamer; performing denaturation of the aptamer to separate the radioactive metal ions from the column; and reusing the column to treat radioactive liquid waste, such that specific metal elements in the radioactive liquid waste may be efficiently isolated while easily separating the radioactive metal ions bound to the column, thereby enabling the column to be reused.

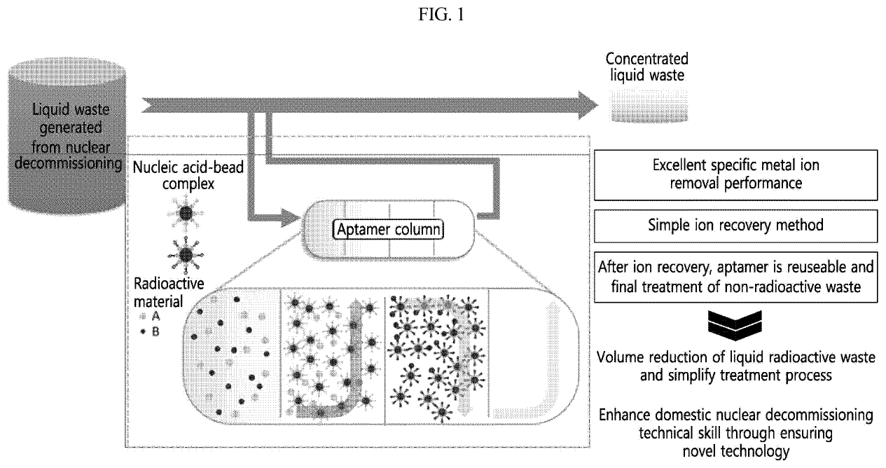

In one embodiment, the method for treating radioactive liquid waste using aptamer according to the present invention is shown in FIG. 1.

The aptamer-bead column of the present invention may include aptamer specifically combined with radioactive metal ions. The aptamer may consist of nucleic acids (ssDNA, RNA) friendly/specifically combinable to target ions. The aptamer may be specifically and strongly combined with specific metal ions while maintaining a stable three-dimensional structure.

The aptamer is not limited to have specific sequence and length so far as it can be specifically combined with the specific metal ion.

The aptamer may consist of 30 to 150 nucleotides (nt). The aptamer may include, for example, 30 to 140 nt, 30 to 130 nt, 30 to 120 nt, 30 to 110 nt, 30 to 100 nt, 30 to 90 nt, 30 to 80 nt, 30 to 70 nt, 30 to 60 nt, 30 to 50 nt, 30 to 40 nt, 40 to 150 nt, 40 to 140 nt, 40 to 130 nt, 40 to 120 nt, 40 to 110 nt, 40 to 100 nt, 40 to 90 nt, 40 to 80 nt, 40 to 70 nt, 40 to 60 nt, 40 to 50 nt, 50 to 150 nt, 50 to 100 nt, 60 to 90 nt, or 60 to 80 nt.

The aptamer has a specific nucleotide sequence in cobalt, nickel, manganese, cesium, etc. present in the radioactive liquid waste and may include DNA, RNA and PNA of a modified nucleic acid type. For example, it may include any one nucleotide sequence among the aptamers shown in Table 1, which have specific sequences in cobalt, nickel and manganese, respectively. Two or more aptamers may be mixed and used. The aptamer may be optimized and used in a range at which its structure and function are not affected, and may include additional sequences at both ends thereof.

The aptamer may include any one nucleotide described in Table 1 below. Two or more aptamers may be mixed and used. The aptamer may be optimized and used in a range at which its structure and function are not affected, and may include additional sequences at both ends thereof.

TABLE 1

| SEQ | | Aptamer | KD value | |
|---|---|---|---|---|
| ID No. | Target | ID | (nM) | Aptamer sequence |
| 1 | Cobalt | FA-C1 | 4.86 ± 6.46 | GGTAATACGACTCACTATAGGGAGATACCAG |
| | | | | CTTATTCAATTTTGCTTGACGAGCCTGTACGT |
| | | | | GGTTCCCTCCAGATGGTCGAGATTGCACTTAC |
| | | | | TATCT |
| 2 | | FA-C2 | 7.73 ± 8.7 | GGTAATACGACTCACTATAGGGAGATACCAG |
| | | | | CTTATTCAATTCACGTTAATCGTATAACTCAC |
| | | | | CTCCTCCTGCTCTTTCTCGAGATTGCACTTAC |
| | | | | TATCT |
| 3 | | FA-C3 | 8.59 ± 6.13 | GGTAATACGACTCACTATAGGGAGATACCAG |
| | | | | CTTATTCAATTTTTGTCCTACGTTGTCTACAT |
| | | | | GGTTCATCTCTTTTTTTCTAGATTGCACTTACT |
| | | | | ATCT |
| 4 | | FA-C4 | 11.1 ± 14.4 | GGTAATACGACTCACTATAGGGAGATACCAG |
| | | | | CTTATTCAATTTGAGTGTTAATGTCTTCGTTT |
| | | | | GCCAGTTTGTTTGAGATTGCACTTACTATCT |
| 5 | | FA-C5 | 13.0 ± 9.86 | GGTAATACGACTCACTATAGGGAGATACCAG |
| | | | | CTTATTCAATTCTAACCCTAGAGTACGTGGTT |
| | | | | TGTCTATGTGTATTCGGGAGATTGCACTTACT |
| | | | | ATCT |
| 6 | | FA-C6 | 20.3 ± 28.8 | GGTAATACGACTCACTATAGGGAGATACCAG |
| | | | | CTTATTCAATTCGCTGCGGTGGTGGGCGGAG |
| | | | | CTATTCCATGTGTACTTCCGAGATTGCACTTA |
| | | | | CTATCT |
| 7 | Nickel | FA-N1 | 6.9 ± 9.5 | GGTAATACGACTCACTATAGGGAGATACCAG |
| | | | | CTTATTCAATTTAGAACGCCCAGTGCATTCCC |
| | | | | CTCCGCTCGACCCCGCATGAGATTGCACTTAC |
| | | | | TATCT |
| 8 | | FA-N2 | 12.3 ± 8.5 | GGTAATACGACTCACTATAGGGAGATACCAG |
| | | | | CTTATTCAATTTCCGCTGCGGTCCGACGATAC |
| | | | | GCTCGGCTCTGTRGTATCGAGATTGCACTTAC |
| | | | | TATCT |
| 9 | | FA-N3 | 13.3 ± 11.9 | GGTAATACGACTCACTATAGGGAGATACCAG |
| | | | | CTTATTCAATTTCCGGTCACGGAGGTAGACTA |
| | | | | CTGTCGTTGGCGGTGGTCGAGATTGCACTTAC |
| | | | | TATCT |
| 10 | | FA-N4 | 18.4 ± 11.5 | GGTAATACGACTCACTATAGGGAGATACCAG |
| | | | | CTTATTCAATTGAGCGCAAGATCTTCCGTAAT |
| | | | | TATGCATAGTGCTCTTTTTGAGATTGCACTTA |
| | | | | CTATCT |
| 11 | | FA-N5 | 19.3 ± 18.5 | GGTAATACGACTCACTATAGGGAGATACCAG |
| | | | | CTTATTCAATTGGCGCGTGCGGAGGCAGGGA |
| | | | | TCGCGGTCGTGATGCCATGGAGATTGCACTT |
| | | | | ACTATCT |
| 12 | | FA-N6 | 20.3 ± 21.7 | GGTAATACGACTCACTATAGGGAGATACCAG |
| | | | | CTTATTCAATTCSTCGGTTTGTGACCCAAAAT |
| | | | | GCGCCGAGGGTACGTTGTGAGATTGCACTTA |
| | | | | CTATCT |
| 13 | Manganese | FA-M1 | 45.64 ± 29.7 | GGTAATACGACTCACTATAGGGAGATACCAG |
| | | | | CTTATTCAATTGGTGGGTGGGTGGGTGGAAG |
| | | | | ATGAAAAGGGGATCCGGGTGAGATTGCACTT |
| | | | | ACTATCT |
| 14 | | FA-M2 | 48.67 ± 31.4 | GGTAATACGACTCACTATAGGGAGATACCAG |
| | | | | CTTATTCAATTCCATCGGTGGGTGGGCGGGT |
| | | | | GGAGAGGGCTTTGTTAAAACAGATTGCACTT |
| | | | | ACTATCT |
| 15 | | FA-M3 | 48.85 ± 25.0 | GGTAATACGACTCACTATAGGGAGATACCAG |
| | | | | CTTATTCAATTAGAGGTCAGGGTAGGGAGGG |
| | | | | GGGAATAAGGTGTCACACGGAGATTGCACTT |
| | | | | ACTATCT |
| 16 | | FA-M4 | 51.17 ± 25.1 | GGTAATACGACTCACTATAGGGAGATACCAG |
| | | | | CTTATTCAATTCGGTGGGTGGGTGGGTGGAG |
| | | | | AAGAGTAAGTGGCGATAAGGAGATTGCACTT |
| | | | | ACTATCT |
| 17 | | FA-M5 | 53.97 ± 26.8 | GGTAATACGACTCACTATAGGGAGATACCAG |
| | | | | CTTATTCAATTTTGATGCACAGTACGGATGAG |
| | | | | TAGGCAGGGTAGGAGTGGAAGATTGCACTTA |
| | | | | CTATCT |
| 18 | | FA-M6 | 58.15 ± 29.0 | GGTAATACGACTCACTATAGGGAGATACCAG |
| | | | | CTTATTCAATTCGGTGGGAGGGCGGGTGGAG |
| | | | | AATTATAATACGATTTGGAAAGATTGCACTT |
| | | | | ACTATCT |

The bead may play a role of passing the radioactive liquid waste through the column for a sufficient time during which the radioactive liquid waste can react with aptamer. The bead is not limited to have specific substances, shapes or sizes if it can play a role as described above.

In one embodiment, the bead may have a spherical shape.

In one embodiment, the bead may be made of polystyrene, latex, agarose, silica or the like.

The bead may have a diameter of 0.5 μm to 1 cm. Within the above range, the bead may have a diameter of, for example, 0.5 μm or more, 1 μm or more, 5 μm or more, 10 μm or more, 50 μm or more, 100 μm or more, 1 mm or more or 5 mm or more. Further, within the above range, the diameter of the bead may be, for example, 5 mm or less, 1 mm or less, 100 μm or less, 50 μm or less, 10 μm or less, 5 μm or less, or 1 μm or less.

With regard to the bead used herein, only beads having the same size may be used, otherwise, two or more types of beads having different sizes from each other may be mixed and used.

The aptamer may be bound to the bead as a fixed phase of the column. The aptamer is bound to the surface of bead through a bond not separated by the radioactive liquid waste. The aptamer may be bound through a chemical bond, antigen-antibody binding, enzyme link or the like. The aptamer may be bound to the bead by a bond of biotin and any one selected from the group consisting of, for example streptavidin, an amine group, a carboxyl group, a hydroxyl group, a sulfone group, a dimethylamine group and an epoxy group.

The aptamer may be bound to the bead in an amount of 0.1 nmol to 10 mmol per 1 ml unit volume of the bead. When a ratio of aptamer and bead is within the above range, it was confirmed that a high removal rate is obtained with optimum binding efficiency and without aptamer to be wasted, thereby attaining economical advantages.

The method for treating radioactive liquid waste using aptamer according to the present invention may include passing radioactive liquid waste including radioactive metal ions through a column filled with aptamer-bound beads to combine metal ions with the aptamer.

The aptamer on the surface of beads may entrap radioactive metal ions as targets, respectively. When the radioactive liquid waste passes through the column repeatedly, the aptamer may not entrap metal ions any more. At this time, the aptamer is subjected to denaturation and the metal ions are separated from the column. In the case of the existing ion exchange resin column, it is difficult to separate metal ions only. However, the column of the present invention can separate metal ions through aptamer denaturation. After separation of metal ions, the column may entrap metal ions again with the aptamer and can be reused. Further, it is also possible to reuse the aptamer only.

Aptamer denaturation refers to deleting a three-dimensional structure from the aptamer. Specifically, if hot water at 50 to 100° C. passes through the column including aptamer, the aptamer may lose the three-dimensional structure and further lose metal ion-specific binding properties.

The hot water used for aptamer denaturation may include the metal ions separated from the column. The hot water may be used for aptamer denaturation repeatedly. When a content of metal ions is more than a standard amount, metal ions in the hot water can be separated and recovered. The hot water after removal of metal ions may be reused for aptamer denaturation (see FIG. 10).

If the metal ions are separated from the column through aptamer denaturation, the denatured aptamer may undergo renaturation at room temperature by adding a buffer to the column.

Hereinafter, the present invention will be described in detail by way of examples.

### EXAMPLE

### 1. Deduction of Optimum Combining Ratio of Aptamer and Bead

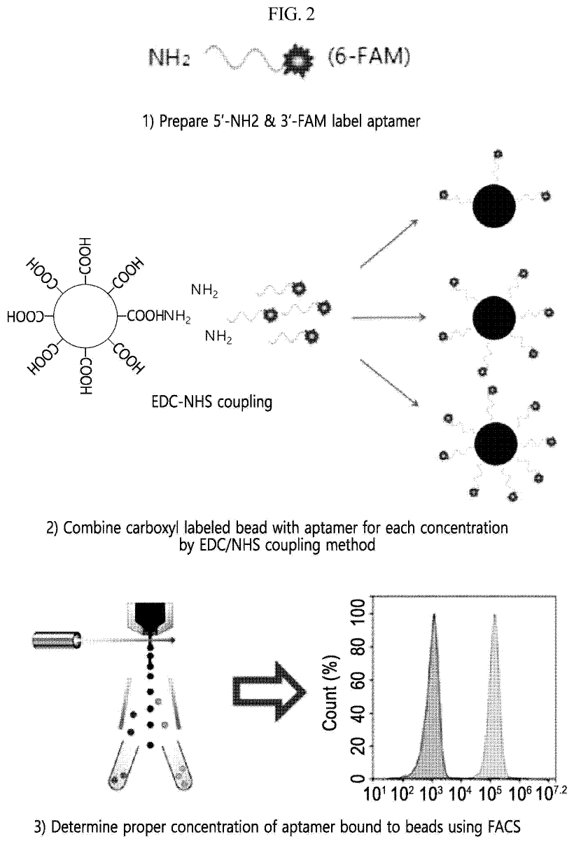

Using 5′—NH₂ & 3′-FAM label aptamer (aptamer FA-C1 of SEQ ID NO: 1) and 5′-biotin & 3′-FAM label aptamer (aptamer of SEQ ID NO: 1), as well as the bead in Table 2 below, the optimum combining ratio of aptamer and bead was determined by the method shown in FIG. 2.

Two types of aptamers, each including an aptamer with binding specificity and 5′-amino modified, 3′-FAM label, were prepared. The prepared aptamer was conjugated to a bead coated with a carboxyl group by EDC/NHS. In consideration of the same bead size during conjugation, different bead conjugate concentrations were applied. The conjugated aptamer-bead complex was measured by a flow cytometer (FACS) to determine desired aptamer-bead concentration suitable for experiments.

TABLE 2

| | Bead 1 | Bead 2 | Bead 3 |
|---|---|---|---|
| Diameter | 0.5 μm | 0.9 μm | 1.4 μm |
| Surface coating | Carboxyl | Carboxyl | Streptavidin |
| Binding aptamer | NH₂-aptamer | NH₂-aptamer | Biotin-aptamer |
| Material of bead | Latex | polystyrene | polystyrene |

Combining ratio of aptamer and bead (bead 2) used in the experiment is shown in Table 3 below.

TABLE 3

| Bead | Diameter | Group | Bead number | Aptamer number |
|---|---|---|---|---|
| Sigma | 0.9 μm | 4 | 1.25 × 10 [symbol] | 4 × ( [symbol] × 10 [symbol] ) |
| [symbol] | | 2 | 1.25 × 10 [symbol] | 2 × ( [symbol] × 10 [symbol] ) |
| | | standard | 1.25 × 10 [symbol] | [symbol] × 10 [symbol] |
| | | ½ | 1.25 × 10 [symbol] | ½ × ( [symbol] × 10 [symbol] ) |
| | | ¼ | 1.25 × 10 [symbol] | ¼ × ( [symbol] × 10 [symbol] ) |
| | | ⅛ | 1.25 × 10 [symbol] | ⅛ × ( [symbol] × 10 [symbol] ) |
| | | 1/16 | 1.25 × 10 [symbol] | 1/16 × ( [symbol] × 10 [symbol] ) |
| | | 1/32 | 1.25 × 10 [symbol] | 1/32 × ( [symbol] × 10 [symbol] ) |
| | | 1/64 | 1.25 × 10 [symbol] | 1/64 × ( [symbol] × 10 [symbol] ) |
| | | 1/128 | 1.25 × 10 [symbol] | 1/128 × ( [symbol] × 10 [symbol] ) |
| | | 1/256 | 1.25 × 10 [symbol] | 1/256 × ( [symbol] × 10 [symbol] ) |

[symbol]

indicates data missing or illegible when filed

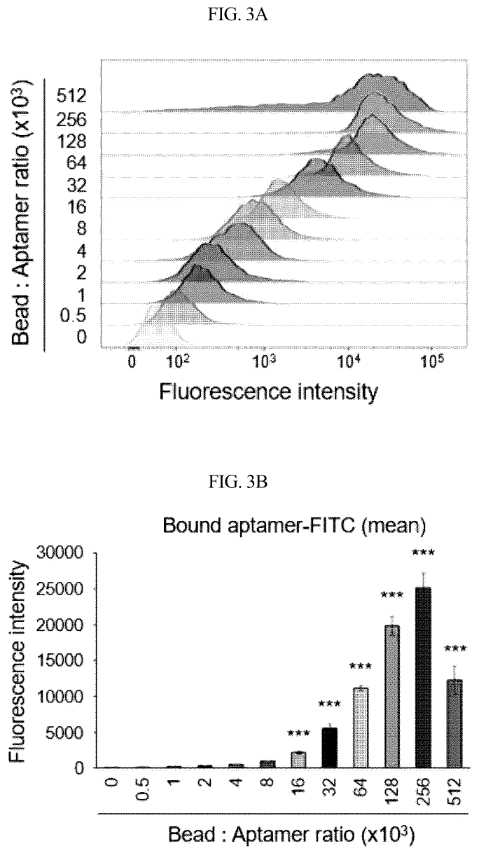

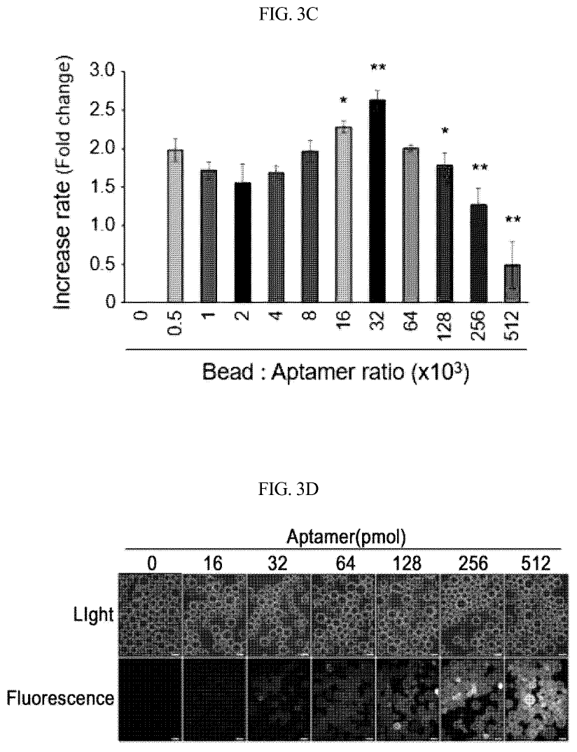

As a result, it could be found that, when the aptamer is bound to the bead in an amount of 0.1 nmol to 10 mmol per 1 ml unit volume of the bead, the best metal ion recovery performance is obtained (FIGS. 3A to 3D).

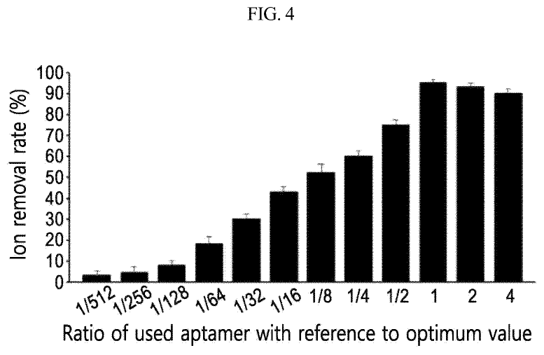

Further, as a result of applying to Co²⁺ aptamer (aptamer FA-C1 of SEQ ID NO: 1) according to the combining ratio in the flow cytometer (FACS) experiment to measure aptamer activity, aptamer in an amount of minimum 1/512 time to maximum 4 times based on the optimum concentration of aptamer obtained from the FACS experiment result was used to the same amount of beads, so as to determine a removal rate of cobalt ions. In view of the result when using the aptamer in the optimum ratio or more, it was confirmed that, if the concentration of aptamer is too high, metal ion recovery performance is reduced due to influence such as interference of aptamer or the like (FIG. 4).

### 2. Evaluation of Performance of Aptamer-Bead Column and Possibility of Reuse Thereof

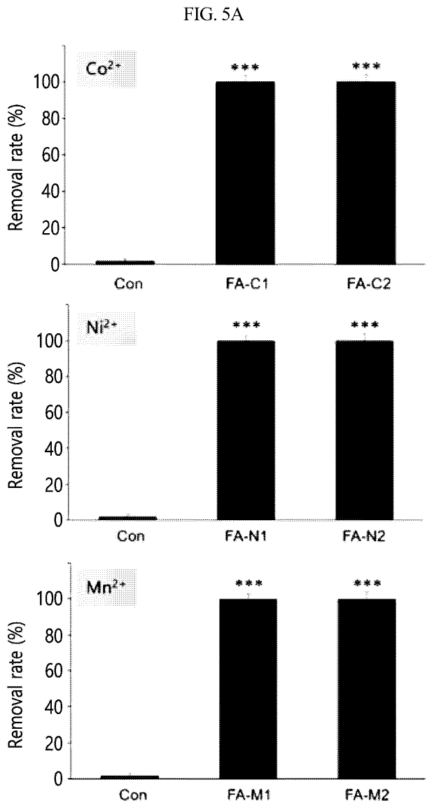

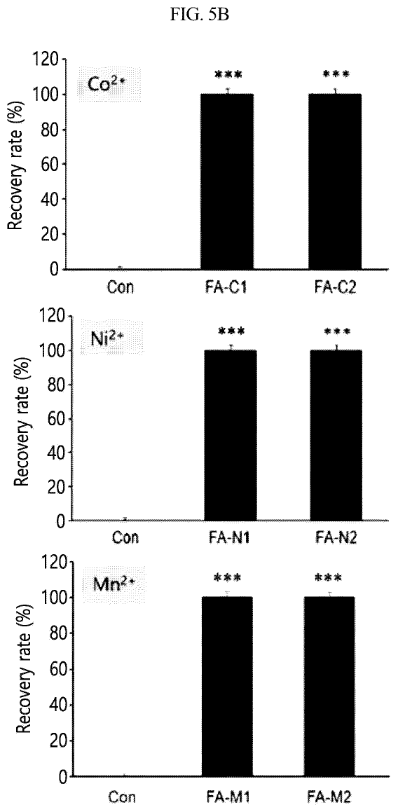

Samples including cobalt, nickel and manganese ions, respectively, in a concentration of 10 μM were passed through a column that includes aptamer (aptamers of SEQ ID NOs: 1, 2, 7, 8, 13 and 14, that is, FA-C1 & C2, FA-N1 & N2, FA-M1 & M2) bound to the beads, followed by measuring concentrations of the metal ions in the obtained solution using an Inductively Coupled Plasma-Optical Emission Spectrometer (ICP-OES) to evaluate the performance of the aptamer-bead column of the present invention. As a result, it was confirmed that all aptamers exhibited 99% or more of target ion removal rate, as shown in FIG. 5A.

Further, when hot water at 95° C. was added to each of the above six (6) different aptamer-bead columns, to which metal ions are combined, in order to perform denaturation of the aptamer, it was confirmed that the metal ions combined with the aptamer were separated from the column and recovered by 99.9% (FIG. 5B). Accordingly, it could be understood that the aptamer and the aptamer-beads column are reusable for entrapping radioactive metal ions.

### 3. Evaluation of the Number of Reuses of Aptamer-Bead Column

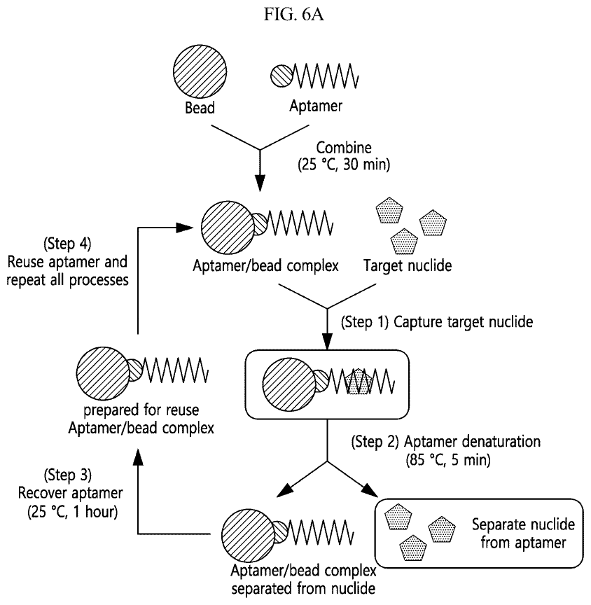

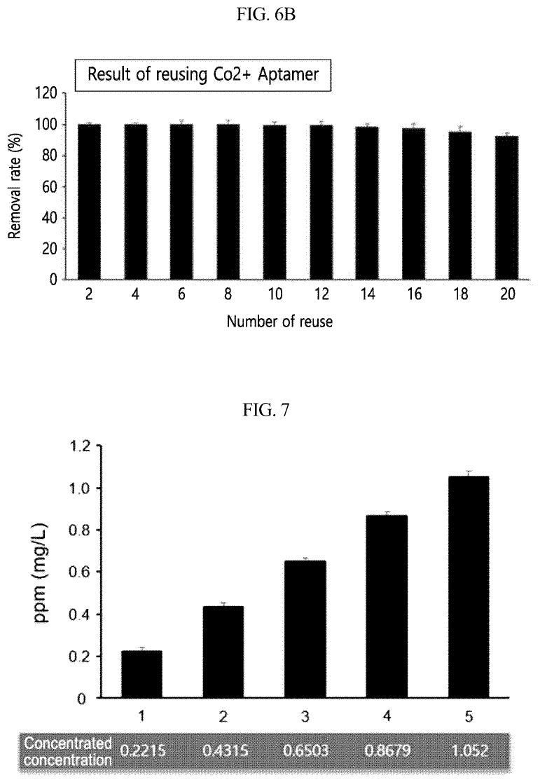

By repeating the above process, how many times the aptamer and the aptamer-beads column could be reused for entrapping radioactive metal ions was investigated as shown in FIG. 6A. As a result, it was confirmed that the removal rate at the first use was 99% or more and substantially equal to those till reusing 12 times as shown in FIG. 6B. However, it was confirmed that, when reused 20 times, the removal rate was decreased to 95% or less.

### 4. Evaluation of Reusability of Hot Water

Whether the hot water used for aptamer denaturation could be reused was investigated. The hot water includes radioactive metal ions and should be disused or discarded as radioactive liquid waste if not reused. Therefore, a hot water concentration experiment reusing the hot water was executed. In particular, cobalt aptamer FA-C1 in Table 1 and 10 UM of cobalt ion sample were subjected to concentration experiments for five times. It was confirmed that the metal ions combined with the aptamer are separated even in the hot water repeatedly reused for five times and concentrated therein (FIG. 7).

### 5. Evaluation of Target Selectivity of Multi-Targeting Aptamer-Bead Column

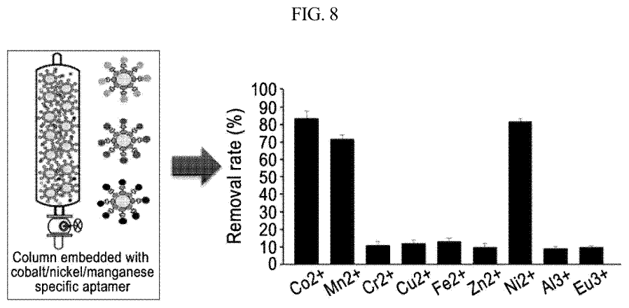

In order to investigate whether the column of the present invention can selectively isolate specific metal ions at a time from radioactive liquid waste containing various metal ions, a sample including 10 UM of nine (9) mixed ions passed through a column filled with cobalt, nickel, manganese, aptamers (aptamers FA-C1, FA-N1 and FA-M1 of SEQ ID NOs: 1, 7 and 13)-bound beads, followed by measuring a concentration of each ion to evaluate the removal rate. As a result, high removal rates of the cobalt, manganese and nickel ions were confirmed such that the column of the present invention was demonstrated to have high target ion selectivity (FIG. 8).

### 6. Field Test

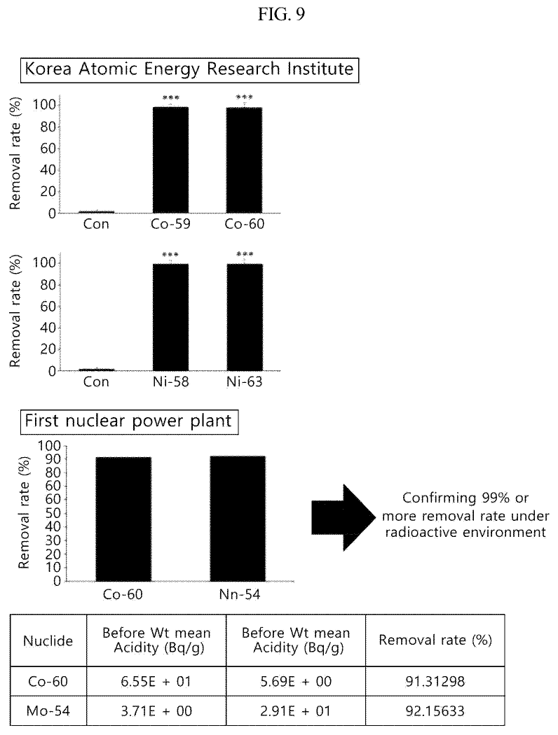

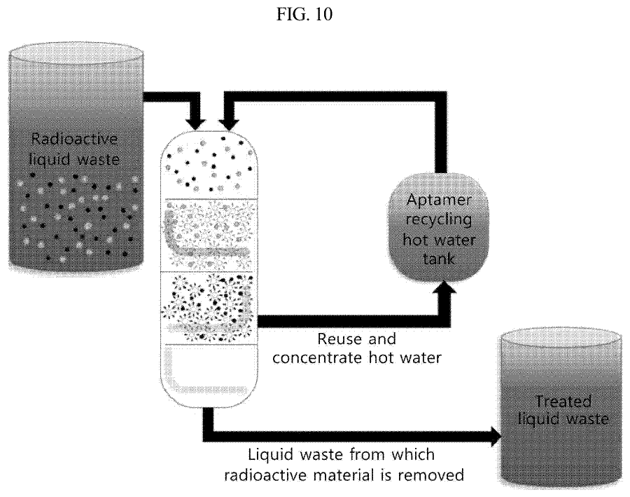

Using actual radioactive liquid waste samples (excluding nickel), a field test was performed. A sample including radioactive material from the Korea Atomic Energy Research Institute and primary cooling water from a nuclear power plant were subjected to the field test. When the above two samples passed through a column filled with beads combined with each of target ion aptamers (aptamers FA-C1 and FA-N1 of SEQ ID NOs: 1 and 7 in the Korea Atomic Energy Research Institute; aptamers FA-C1, FA-N1 and FA-M1 of SEQ ID NOs: 1, 7 and 13 in the nuclear power plant), it was confirmed that 90% or more radioactive metal ions were removed (FIG. 9).

What is claimed is:

1. A method for treating radioactive liquid waste using aptamer, the method comprising:
passing radioactive liquid waste including radioactive metal ions through a column filled with aptamer-bound beads to combine the metal ions with the aptamer;
performing denaturation of the aptamer to separate the radioactive metal ions from the column; and
reusing the column to treat radioactive liquid waste.

2. The method according to claim 1, wherein the denaturation is performed by passing hot water at 50 to 100° C. through the column.

3. The method according to claim 2, further comprising denaturation of the aptamer by reusing the hot water.

4. The method according to claim 1, further comprising renaturation of the denaturated aptamer at room temperature by adding a buffer to the column.

5. The method according to claim 1, wherein the aptamer having a length of 30 to 150 nt.

6. The method according to claim 1, wherein the aptamer has any one nucleotide sequence of SEQ ID NOs: 1 to 18.

7. The method according to claim 1, wherein the bead has a diameter of 0.5 μm to 1 cm.

8. The method according to claim 1, wherein the aptamer is bound to the bead in an amount of 0.1 nmol to 10 mmol per 1 ml unit volume of the bead.

9. The method according to claim 1, wherein the aptamer is bound to the bead by a bond of biotin and any one selected from the group consisting of streptavidin, an amine group, a carboxyl group, a hydroxyl group, a sulfone group, a dimethylamine group and an epoxy group.

10. An aptamer-bead column for treating radioactive liquid waste, comprising:
a column;
beads having a diameter of 0.5 μm to 1 cm filled the column;
aptamer having a length of 30 to 150 nt, the aptamer bound to the beads.

11. The aptamer-bead column according to claim 10, wherein the aptamer is bound to the bead by a bond of biotin and any one selected from the group consisting of streptavidin, an amine group, a carboxyl group, a hydroxyl group, a sulfone group, a dimethylamine group and an epoxy group.

12. The aptamer-bead column according to claim 10, wherein the aptamer is bound to the bead in an amount of 0.1 nmol to 10 mmol per 1 ml unit volume of the bead.

13. The aptamer-bead column according to claim 10, wherein the aptamer has any one nucleotide sequence of SEQ ID NOs: 1 to 18.


SEQUENCE LISTING (18 sequences)

SEQ ID NO: 1 - DNA, 100 bp, synthetic construct
  source 1..100: mol_type = other DNA
ggtaatacgactcactatagggagataccagcttattcaattttgcttgacgagcctgtacgtggttccctccagatggtcgagattgcacttactatct

SEQ ID NO: 2 - DNA, 100 bp, synthetic construct
  source 1..100: mol_type = other DNA
ggtaatacgactcactatagggagataccagcttattcaattcacgttaatcgtataactcacctcctcctgctctttctcgagattgcacttactatct

SEQ ID NO: 3 - DNA, 100 bp, synthetic construct
  source 1..100: mol_type = other DNA
ggtaatacgactcactatagggagataccagcttattcaatttttgtcctacgttgtctacatggttcatctctttttttctagattgcacttactatct

SEQ ID NO: 4 - DNA, 94 bp, synthetic construct
  source 1..94: mol_type = other DNA
ggtaatacgactcactatagggagataccagcttattcaatttgagtgttaatgtcttcgtttgccagtttgtttgagattgcacttactatct

SEQ ID NO: 5 - DNA, 99 bp, synthetic construct
  source 1..99: mol_type = other DNA
ggtaatacgactcactatagggagataccagcttattcaattctaaccctagagtacgtggtttgtctatgtgtattcgggagattgcacttactatct

SEQ ID NO: 6 - DNA, 100 bp, synthetic construct
  source 1..100: mol_type = other DNA
ggtaatacgactcactatagggagataccagcttattcaattcgctgcggtggtgggcggagctattccatgtgtacttccgagattgcacttactatct

SEQ ID NO: 7 - DNA, 100 bp, synthetic construct
  source 1..100: mol_type = other DNA
ggtaatacgactcactatagggagataccagcttattcaatttagaacgcccagtgcattcccctccgctcgaccccgcatgagattgcacttactatct

SEQ ID NO: 8 - DNA, 100 bp, synthetic construct
  source 1..100: mol_type = other DNA
ggtaatacgactcactatagggagataccagcttattcaatttccgctgcggtccgacgatacgctcggctctgtrgtatcgagattgcacttactatct

SEQ ID NO: 9 - DNA, 100 bp, synthetic construct
  source 1..100: mol_type = other DNA
ggtaatacgactcactatagggagataccagcttattcaatttccggtcacggaggtagactactgtcgttggcggtggtcgagattgcacttactatct

SEQ ID NO: 10 - DNA, 101 bp, synthetic construct
  source 1..101: mol_type = other DNA
ggtaatacgactcactatagggagataccagcttattcaattgagcgcaagatcttccgtaattatgcatagtgctctttttgagattgcacttactatct

SEQ ID NO: 11 - DNA, 100 bp, synthetic construct
  source 1..100: mol_type = other DNA
ggtaatacgactcactatagggagataccagcttattcaattggcgcgtgcggaggcagggatcgcggtcgtgatgccatggagattgcacttactatct

SEQ ID NO: 12 - DNA, 100 bp, synthetic construct
  source 1..100: mol_type = other DNA
ggtaatacgactcactatagggagataccagcttattcaattcstcggtttgtgacccaaaatgcgccgagggtacgttgtgagattgcacttactatct

SEQ ID NO: 13 - DNA, 100 bp, synthetic construct
  source 1..100: mol_type = other DNA
ggtaatacgactcactatagggagataccagcttattcaattggtgggtgggtgggtggaagatgaaaaggggatccgggtgagattgcacttactatct

SEQ ID NO: 14 - DNA, 100 bp, synthetic construct
  source 1..100: mol_type = other DNA
ggtaatacgactcactatagggagataccagcttattcaattccatcggtgggtgggcgggtggagagggctttgttaaaacagattgcacttactatct

SEQ ID NO: 15 - DNA, 100 bp, synthetic construct
  source 1..100: mol_type = other DNA
ggtaatacgactcactatagggagataccagcttattcaattagaggtcagggtagggaggggggaataaggtgtcacacggagattgcacttactatct

SEQ ID NO: 16 - DNA, 100 bp, synthetic construct
  source 1..100: mol_type = other DNA
ggtaatacgactcactatagggagataccagcttattcaattcggtgggtgggtgggtggagaagagtaagtggcgataaggagattgcacttactatct

SEQ ID NO: 17 - DNA, 100 bp, synthetic construct
  source 1..100: mol_type = other DNA
ggtaatacgactcactatagggagataccagcttattcaattttgatgcacagtacggatgagtaggcagggtaggagtggaagattgcacttactatct

SEQ ID NO: 18 - DNA, 100 bp, synthetic construct
  source 1..100: mol_type = other DNA
ggtaatacgactcactatagggagataccagcttattcaattcggtgggagggcgggtggagaattataatacgatttggaaagattgcacttactatct